SEQUENCE LISTING (1 sequence)

SEQ ID NO: 1 - protein, 219 aa, Artificial Sequence
  Description of Artificial Sequence Synthetic polypeptide
IPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAQSKPERRRGLSWKSQNGRLYSIKSTKMLIHPTDSESFE

(19) United States
(12) Patent Application Publication
(10) Pub. No.: US 2024/0238376 A1
(43) Pub. Date: Jul. 18, 2024

(54) METHODS FOR THE TREATMENT OF OSTEOARTHRITIS

(71) Applicant: Novartis AG, Basel (CH)

(72) Inventors: Matthias Klaus SCHIEKER, Munich (DE); Celeste SCOTTI, Basel (CH)

(21) Appl. No.: 18/562,108

(22) PCT Filed: May 23, 2022
(86) PCT No.: PCT/IB2022/054817

§ 371 Date: Nov. 17, 2023

Related U.S. Application Data
(60) Provisional application No. 63/192,303, filed on May 24, 2021.

(51) Int. Cl. A61K 38/18; A61K 9/00; A61K 39/00; A61K 39/395; A61P 19/02; C07K 16/24

(52) U.S. Cl. CPC A61K 38/1891; A61K 9/0019; A61K 39/3955; A61P 19/02; C07K 16/245; A61K 2039/505; A61K 2039/545; C07K 2317/76

(57) ABSTRACT

Provided herein are methods and dosage regimens for the treatment of osteoarthritis (e.g., knee osteoarthritis). These methods and dosage regimens include intra-articular injections of Compound 1 alone, or in combination with an anti-inflammatory antibody (e.g., an anti-IL-10 antibody).

### SEQUENCE LISTING

The instant application contains a Sequence Listing which has been submitted electronically in ASCII format and is hereby incorporated by reference in its entirety. Said ASCII copy, created on May 18, 2022, is named PAT059121-WO-PCT_SL.txt and is 2,266 bytes in size.

### TECHNICAL FIELD

The disclosure relates to methods, treatment regimens, uses, kits and therapies for treating osteoarthritis by administering a therapeutic polypeptide alone or in combination with an anti-inflammatory antibody, such as an anti-IL-1β antibody.

### BACKGROUND OF THE INVENTION

Osteoarthritis (OA), a slowly progressive disease with a multifactorial pathophysiology, is one of the most common chronic health conditions in adults, and a leading cause of pain and disability (OARSI 2016, submitted to the U.S. Food and Drug Administration, viewed 16 Dec. 2019, p. 1-103). Due to the demographic changes in an ageing society and an increase in the incidence of obesity, the prevalence of OA will steadily increase, posing a significant burden on global health-care systems. (OARSI 2016; Fu & Griffin 2014, Biomaterials, vol 16. Springer, Ch). On the individual level OA leads to suffering with OA symptoms being responsible for 6.8% of overall DALYs (disability adjusted life years). (Cross et al 2014, Ann. Rheum. Dis. 73, 1323-1330; Kassebaum et al 2016, Lancet 388, 1603-1658). An excess mortality rate from cardiovascular events also has been observed in association with OA. (Kloppenburg & Berenbaum 2019, Osteoarthr. Cartil. 28, 242-248). Finally, OA is a relevant financial risk factor with loss of work days and out of pocket expenditure. (Puig-Junoy & Zamora 2015, Semin. Arthritis Rheum. 44, 531-541; Sharif et al 2015, Osteoarthr. Cartil. October 23 (10): 1654-63).

The knee joint is the most common weight bearing joint affected by OA. Current medical treatments of OA focus on addressing pain, but there are no disease-modifying OA drugs (DMOADs), e.g., chondro-anabolic treatments, available to induce cartilage regeneration. (Lohmander and Roos 2019, Nat Rev Rheumatol p. 133-135). Eventual joint failure requiring surgical joint replacement is common, with over a million such operations annually in the United States (Williams et al 2015, NCHS Data Brief p. 1-8; Wolford et al 2015, NCHS Data Brief p. 1-8). However, not all patients are satisfied with the result, or benefit from joint replacement surgery. In a long-term outcome study in an OA cohort with a high prevalence of multiple affected joints and comorbidities, only half of those who received joint replacement achieved a good surgical outcome, defined as improved pain and reduced disability. (Hawker et al 2013, Arthritis Rheum. 65(5):1243-52).

Intra-articular (“i.a.”) inflammation is present in 30-50% of OA patients with moderate-severe disease in acute flare phases of cartilage degradation and/or as chronic low-grade inflammation. Moreover, inflammation is actively involved in the pathophysiology of OA and the progression of the disease even in the absence of overt symptoms. Especially in later stages of OA, intra-articular inflammation is common. Intra-articular corticosteroids (IACS) is the current standard of care for inflamed OA joints, but reviews of its efficacy indicate there is a high level of uncertainty as to the benefit. (Orchard 2020, BMJ 368: 16923). In addition, IACS have been shown to have long-term detrimental effects on articular cartilage, accelerating disease progression. There remains a need for dosing regimens for therapies that address this significant patient population.

### SUMMARY OF THE INVENTION

The methods of the present invention comprise administering to a subject an effective amount of a modified human ANGPTL3 polypeptide, i.e., Compound 1, according to the dosing regimens disclosed herein. In certain embodiments, the present invention also provides for intra-articular administration of an effective amount of an anti-IL-1β antibody, wherein said anti-IL-1B may be administered before, after, or concurrently with Compound 1.

In some embodiments, the present invention provides a method of treating OA, the method comprising administering to a human subject in need thereof one or more doses of a therapeutically effective amount of Compound 1 by intra-articular injection to a joint (e.g., knee joint) of the subject according to a dosing regimen comprising one or more dosing cycles. In certain embodiments, the OA is knee OA. In other embodiments the OA is with intra-articular inflammation (e.g., knee OA with intra-articular inflammation). In some embodiments, the dosing cycle is a six-month dosing cycle comprising three intra-articular injections per dosing cycle with one injection administered once a month for three consecutive months. In certain embodiments, the dosing regimen comprises at least two dosing cycles. In some embodiments, the amount of Compound 1 administered per dose is 40 mg. In other embodiments, treatment according to said dosing regimen results in the maintenance or regeneration of articular cartilage tissue as determined by magnetic resonance imaging (MRI) analysis.

In some embodiments, the present invention provides a method of treating knee OA with intra-articular inflammation, the method comprising administering to a human subject in need thereof one or more doses of a therapeutically effective amount of an anti-inflammatory antibody by intra-articular injection to a joint (e.g., knee joint) of the subject. In some embodiments, the amount of the anti-inflammatory antibody administered per dose is 600 mg. In some embodiments, the anti-inflammatory antibody is an anti-IL-1β antibody. In certain embodiments, the anti-inflammatory antibody is canakinumab. In some embodiments, administration of the anti-inflammatory antibody results in a reduction in OA pain.

In some embodiments, the present invention provide a method of treating knee OA with intra-articular inflammation, the method comprising administering to a human subject in need thereof (a) a dose of about 600 mg of an anti-IL-1β antibody, and (b) one or more doses of a therapeutically effective amount of Compound 1 according to a dosing regimen comprising one or more dosing cycles, wherein said dose of the anti-IL-1β antibody and said one or more doses of a therapeutically effective amount of Compound 1 are administered to a joint (e.g., knee joint) of the subject by intra-articular administration. In some embodiments, the dose of the anti-IL-1β antibody is administered prior to beginning the Compound 1 dosing regimen. In other embodiments, the anti-IL-1β antibody is administered two weeks or four weeks prior to beginning the Compound 1 dosing regimen. In some embodiments, the dosing cycle is a six-month dosing cycle comprising three intra-articular injections per dosing cycle with one injection administered once a month for three consecutive months. In certain embodiments, the dosing regimen comprises at least two dosing cycles. In certain embodiments, a dose of 600 mg of an anti-IL-1β antibody is administered prior to beginning the second dosing cycle of Compound 1. In some embodiments, the amount of Compound 1 administered per dose is 40 mg. In other embodiments, treatment according to said dosing regimen results in the maintenance or regeneration of articular cartilage tissue as determined by MRI analysis. In certain other embodiments, administration of the anti-IL-1β antibody and Compound 1 results in a reduction in OA pain.

In other embodiments, the present invention provides Compound 1 for use in the treatment of OA either alone or in a combination therapy with an anti-IL-1β antibody. For example, such use is via administration to a human subject in need thereof of one or more doses of a therapeutically effective amount of Compound 1 by intra-articular injection to a joint (e.g., knee joint) of the subject according to a dosing regimen comprising one or more dosing cycles. In certain embodiments, the OA is knee OA. In other embodiments the OA is with intra-articular inflammation (e.g., knee OA with intra-articular inflammation). In some embodiments, the dosing cycle is a six-month dosing cycle comprising three intra-articular injections per dosing cycle with one injection administered once a month for three consecutive months. In certain embodiments, the dosing regimen comprises at least two dosing cycles. In some embodiments, the amount of Compound 1 administered per dose is 40 mg. In other embodiments, treatment according to said dosing regimen results in the maintenance or regeneration of articular cartilage tissue as determined by MRI analysis.

In other embodiments, the present invention provides an anti-inflammatory antibody for use in the treatment of OA with intra-articular inflammation either alone or in a combination therapy with an anti-IL-1β antibody. For example, such use is via administration to a human subject in need thereof one or more doses of a therapeutically effective amount of an anti-inflammatory antibody by intra-articular injection to a joint (e.g., knee joint) of the subject. In some embodiments, the amount of the anti-inflammatory antibody administered per dose is 600 mg. In some embodiments, the anti-inflammatory antibody is an anti-IL-1β antibody. In certain embodiments, the anti-inflammatory antibody is canakinumab. In some embodiments, administration of the anti-inflammatory antibody results in a reduction in OA pain.

In other embodiments, the present invention provides Compound 1 and an anti-IL-1β antibody for use in the treatment of OA with intra-articular inflammation. For example, such combination for use is via administration to a human subject in need thereof (a) a dose of about 600 mg of an anti-IL-1β antibody, and (b) one or more doses of a therapeutically effective amount of Compound 1 according to a dosing regimen comprising one or more dosing cycles, wherein said dose of the anti-IL-1β antibody and said one or more doses of a therapeutically effective amount of Compound 1 are administered by intra-articular administration to a joint (e.g., knee joint) of the subject. In some embodiments, the dose of the anti-IL-1 antibody is administered prior to beginning the Compound 1 dosing regimen. In other embodiments, the anti-IL-1β antibody is administered two weeks or four weeks prior to beginning the Compound 1 dosing regimen. In some embodiments, the dosing cycle is a six-month dosing cycle comprising three intra-articular injections per dosing cycle with one injection administered once a month for three consecutive months. In certain embodiments, the dosing regimen comprises at least two dosing cycles. In certain embodiments, a dose of about 600 mg of an anti-IL-1β antibody is administered prior to beginning the second dosing cycle of Compound 1. In some embodiments, the amount of Compound 1 administered per dose is 40 mg. In other embodiments, treatment according to said dosing regimen results in the maintenance or regeneration of articular cartilage tissue as determined by MRI analysis. In certain other embodiments, administration of the anti-IL-1β antibody and Compound 1 results in a reduction in OA pain.

In other embodiments, the present invention provides Compound 1 for use in the manufacture of a medicament for the treatment of OA, either alone or in a combination therapy with a medicament comprising an anti-IL-1β antibody. For example, such use is via administration to a human subject in need thereof one or more doses of a therapeutically effective amount of Compound 1 by intra-articular injection to a joint (e.g., knee joint) of the subject according to a dosing regimen comprising one or more dosing cycles. In certain embodiments, the OA is knee OA. In other embodiments the OA is with intra-articular inflammation (e.g., knee OA with intra-articular inflammation). In some embodiments, the dosing cycle is a six-month dosing cycle comprising three intra-articular injections per dosing cycle with one injection administered once a month for three consecutive months. In certain embodiments, the dosing regimen comprises at least two dosing cycles. In some embodiments, the amount of Compound 1 administered per dose is 40 mg. In other embodiments, treatment according to said dosing regimen results in the maintenance or regeneration of articular cartilage tissue as determined by MRI analysis.

In other embodiments, the present invention provides an anti-inflammatory antibody for use in the manufacture of a medicament for the treatment of OA with intra-articular inflammation, either alone or in a combination therapy with a medicament comprising an anti-IL-1β antibody. For example, such use is via administration to a human subject in need thereof one or more doses of a therapeutically effective amount of an anti-inflammatory antibody by intra-articular injection to a knee joint of the subject. In some embodiments, the amount of the anti-inflammatory antibody administered per dose is 600 mg. In some embodiments, the anti-inflammatory antibody is an anti-IL-1β antibody. In certain embodiments, the anti-inflammatory antibody is canakinumab. In some embodiments, administration of the anti-inflammatory antibody results in a reduction in OA pain.

In other embodiments, the present invention provides Compound 1 and an anti-IL-1β antibody for use in the manufacture of a medicament for the treatment of OA with intra-articular inflammation. For example, such use is via administration to a human subject in need thereof (a) a dose of about 600 mg of an anti-IL-1β antibody, and (b) one or more doses of a therapeutically effective amount of Compound 1 according to a dosing regimen comprising one or more dosing cycles, wherein said dose of the anti-IL-1β antibody and said one or more doses of a therapeutically effective amount of Compound 1 are administered by intra-articular administration to a joint (e.g., knee joint) of the subject. In some embodiments, the dose of the anti-IL-1β antibody is administered prior to beginning the Compound 1 dosing regimen. In other embodiments, the anti-IL-1β antibody is administered two weeks or four weeks prior to beginning the Compound 1 dosing regimen. In some embodiments, the dosing cycle is a six-month dosing cycle comprising three intra-articular injections per dosing cycle with one injection administered once a month for three consecutive months. In certain embodiments, the dosing regimen comprises at least two dosing cycles. In certain embodiments, a dose of about 600 mg of an anti-IL-1β antibody is administered prior to beginning the second dosing cycle of Compound 1. In some embodiments, the amount of Compound 1 administered per dose is 40 mg. In other embodiments, treatment according to said dosing regimen results in the maintenance or regeneration of articular cartilage tissue as determined by MRI analysis. In certain other embodiments, administration of the anti-IL-1β antibody and Compound 1 results in a reduction in OA pain.

In other embodiments, the present invention provides a pharmaceutical composition comprising Compound 1 for use in the treatment of OA, either alone or in a combination therapy with a medicament comprising an anti-IL-1β antibody. For example, such use is via administration to a human subject in need thereof one or more doses of a therapeutically effective amount of Compound 1 by intra-articular injection to a joint (e.g., knee joint) of the subject according to a dosing regimen comprising one or more dosing cycles. In certain embodiments, the OA is knee OA. In other embodiments the OA is with intra-articular inflammation (e.g., knee OA with intra-articular inflammation). In some embodiments, the dosing cycle is a six-month dosing cycle comprising three intra-articular injections per dosing cycle with one injection administered once a month for three consecutive months. In certain embodiments, the dosing regimen comprises at least two dosing cycles. In some embodiments, the amount of Compound 1 administered per dose is 40 mg. In other embodiments, treatment according to said dosing regimen results in the maintenance or regeneration of articular cartilage tissue as determined by MRI analysis.

In other embodiments, the present invention provides a pharmaceutical composition comprising an anti-inflammatory antibody for use in the treatment of OA with intra-articular inflammation, either alone or in combination with Compound 1. For example, such use is via administration to a human subject in need thereof one or more doses of a therapeutically effective amount of an anti-inflammatory antibody by intra-articular injection to a knee joint of the subject. In some embodiments, the amount of the anti-inflammatory antibody administered per dose is 600 mg. In some embodiments, the anti-inflammatory antibody is an anti-IL-1β antibody. In certain embodiments, the anti-inflammatory antibody is canakinumab. In some embodiments, administration of the anti-inflammatory antibody results in a reduction in OA pain.

In other embodiments, the present invention provides a pharmaceutical composition comprising Compound 1 and a pharmaceutical composition comprising an anti-IL-1β antibody for the treatment of OA with intra-articular inflammation via administration to a human subject in need thereof (a) a dose of about 600 mg of an anti-IL-1β antibody, and (b) one or more doses of a therapeutically effective amount of Compound 1 according to a dosing regimen comprising one or more dosing cycles, wherein said dose of the anti-IL-1β antibody and said one or more doses of a therapeutically effective amount of Compound 1 are administered by intra-articular administration to a joint (e.g., knee joint) of the subject. In some embodiments, the dose of the anti-IL-1β antibody is administered prior to beginning the Compound 1 dosing regimen. In other embodiments, the anti-IL-1β antibody is administered two weeks or four weeks prior to beginning the Compound 1 dosing regimen. In some embodiments, the dosing cycle is a six-month dosing cycle comprising three intra-articular injections per dosing cycle with one injection administered once a month for three consecutive months. In certain embodiments, the dosing regimen comprises at least two dosing cycles. In certain embodiments, a dose of about 600 mg of an anti-IL-1β antibody is administered prior to beginning the second dosing cycle of Compound 1. In some embodiments, the amount of Compound 1 administered per dose is 40 mg. In other embodiments, treatment according to said dosing regimen results in the maintenance or regeneration of articular cartilage tissue as determined by MRI analysis. In certain other embodiments, administration of the anti-IL-1β antibody and Compound 1 results in a reduction in OA pain.

Various aspects of the disclosure are described herein and in the claims.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise. Although methods and materials similar or equivalent to those described herein can be used in the practice or testing of the disclosure, suitable methods and materials are described below. All publications, patent applications, patents, and other references mentioned herein are incorporated by reference in their entireties for all purposes. The references cited herein are not admitted to be prior art to the claimed disclosure. In the case of conflict, the present specification, including definitions, will control. In addition, the materials, methods, and examples are illustrative only and are not intended to be limiting.

Other features and advantages of compounds, compositions, and methods disclosed herein will be apparent from the following detailed description and claims

### DESCRIPTION OF THE DRAWINGS

FIG. 1 is a schematic representation of dosing regimens. CMPD1=Compound 1.

FIG. 2 depicts immunohistochemical staining of osteochondral knee tissue obtained during a total knee replacement demonstrating that Compound 1 has a more pronounced penetration in (A) damaged cartilage tissue versus (B) undamaged cartilage tissue.

FIG. 3 depicts RNA-Seq analysis of cartilage biopsies harvested from worn and unworn areas of the knee demonstrates that following intra-articular injection of Compound 1: A) 151 genes were significantly up- or down-regulated in response to Compound 1 as compared to placebo, and B) several genes involved in cartilage homeostasis and repair are modulated by Compound 1 up to 21 days post-injection. CMPD1=Compound 1.

FIG. 4A) a cartilage 3D MRI rendering showing administration of a single intra-articular injection of Compound 1 to an exemplary patient undergoing autologous chondrocyte implantation resulted in an increased filling of a surgically created lesion at the donor site as compared to placebo. B) is a graphical representation demonstrating that percent refilling of the donor site is increased over time in an exemplary patient administered a single intra-articular injection of Compound 1 as compared to placebo. CMPD1=Compound 1.

FIG. 5A) Sodium-MRI images confirming the hyaline-like cartilage nature of regenerated tissue at the donor site of an exemplary patient undergoing autologous chondrocyte implantation that was administered a single intra-articular injection of Compound 1 at 2 time points as compared to placebo. B) demonstrates that the treatment response calculated as percent change from baseline in sodium signal intensity in the donor site corrected by sodium signal intensity in the reference region of an exemplary patient administered Compound 1 increases over time as compared to placebo. CMPD1=Compound 1.

FIG. 6 depicts 3D MRI images from an exemplary patient treated with four weekly intra-articular injections of Compound 1 demonstrating successful articular cartilage lesion filling. CMPD1=Compound 1.

FIG. 7 depicts the model estimated percentage difference of Compound 1 to placebo in cartilage defect volume over time (PD analysis set) in patients with partial thickness lesions demonstrating that Compound 1 decreases the cartilage defect volume. A) overall and B) in femur. MMRM=mixed model repeated measures; CMPD1=Compound 1.

FIG. 8 depicts arithmetic mean (SD) serum concentration-time profiles in knee OA patients after single i.a. dose of 150, 300 or 600 mg canakinumab in the SAD study.

### DETAILED DESCRIPTION OF THE INVENTION

Modified human ANGPTL3 polypeptides have been shown to demonstrate chondrogenic and chondroprotective effects. Examples of such polypeptides have been previously described in WO2014/138687, the contents of which are fully incorporated by reference. Methods of administering such polypeptides for the purpose of treating cartilage damage and/or arthritis have been previously described in WO2018/087727. The methods disclosed in WO2018/087727 include intra-articular injections on a weekly or monthly basis until such time as the cartilage damage or arthritis has been treated. However, intra-articular injections are invasive, and there remains a need to develop a dosing regimen that provides a therapeutic benefit to patients while minimizing the number of intra-articular injections both to lessen patient discomfort and to reduce the potential for inadvertent joint damage or injection-related infections. The prior disclosures also do not address the effectiveness of a modified human ANGPTL3 polypeptide in treating symptomatic knee OA patients with intra-articular inflammation alone or in combination with another therapeutic agent, e.g., an anti-IL-1β antibody. Thus, there remains a need for dosing regimens for therapies that address this significant patient population.

The inventors have surprisingly discovered that intra-articular administration of Compound 1 is effective to reduce symptoms and rebuild cartilage structure in symptomatic OA patients. In some embodiments, the OA is knee OA with or without intra-articular inflammation. In some embodiments, intra-articular administration of Compound 1 is before, after, or concurrent to intra-articular administration of an anti-IL1-β antibody.

### Definitions

The term “subject” refers to an animal, human or non-human, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

The term “treatment”, “treating,” or “treat” is herein defined as therapeutic measures for the reduction or amelioration of the progression, severity and/or duration of an undesired physiological change or disorder (e.g., an arthritic disease (e.g., OA)), or the amelioration of one or more symptoms (preferably, one or more discernible symptoms) of the disorder resulting from the administration of one or more therapeutic agents. In other embodiments, the terms “treatment,” “treating,” or “treat” refer to the reduction or stabilization of the progression of a disorder, such as OA, either physically by, e.g., reduction or stabilization of a discernible symptom, physiologically by, e.g., reduction or stabilization of a physical parameter, or both. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and reversal (whether partial or total), whether detectable or undetectable.

A subject is “in need of” a treatment if such subject would benefit biologically, medically or in quality of life from such treatment.

The term “pharmaceutically acceptable” as used herein refers to those compounds, materials, compositions and/or dosage forms, which are, within the scope of sound medical judgment, suitable for contact with the tissues of a subject, e.g., a mammal or human, without excessive toxicity, irritation, allergic response and other problems or complications commensurate with a reasonable benefit/risk ratio.

The term “administration” or “administering” of the subject compound means providing a drug, a modified derivative of a drug, or a prodrug to a subject in need of treatment.

The term “dosing regimen,” as used herein, refers to the treatment plan specifically indicating the administration pattern of a drug over a period of time. The dosing regimen defines the amount of a drug and the number and frequency of its administrations that is employed in the treatment of a disease. The dosing regimens of the present invention may comprise one or more dosing cycles.

The term “dosing cycle,” as used herein, means administering a drug for a period of time (i.e., the dosing period) followed by a resting period before administration of the drug is resumed. A dosing cycle begins with the first administration of the drug in that cycle. The term “resting period,” as used herein, refers to a period of time during which the subject is not given a drug (i.e., a period of time wherein the treatment with a drug is withheld). For example, if a drug is given on a daily, weekly, or monthly basis, there would be rest period if the administration is discontinued for some time, e.g., for some number of days, weeks, or months. The dosing period and/or the resting period of the dosing cycle can be the same or different between cycles. For example, if the dosing period is once weekly the resting period may be one week or more than one week. It is further contemplated that the dose of the drug administered can be the same or different between cycles.

The term “dose” refers to a specified amount of a drug administered at one time. As used herein, the dose is the amount of the drug that elicits a therapeutic effect.

The term “a therapeutically effective amount” of a drug refers to an amount of a drug that will elicit the desired biological or medical response in a subject, for example, at least partially ameliorate symptoms, alleviate conditions, slow or delay progression, or reverse a disorder or disease.

As used herein, the terms “a” and “an” and “the” and similar references in the context of describing the invention are to be construed to cover both the singular and the plural, unless otherwise indicated herein or clearly contradicted by context. Where the plural form is used for compounds, salts, and the like, this is taken to mean also a single compound, salt, or the like.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When describing a dosage herein as “about” a specified amount, the actual dosage can vary by up to 10% from the stated amount: this usage of “about” recognizes that the precise amount in a given dosage form may differ slightly from an intended amount for various reasons without materially affecting the in vivo effect of the administered compound.

When describing a dosage herein as a specified amount, i.e. without the term “about”, the actual dosage can vary by up to 10% (preferably by up to 5%) from the stated amount: this usage recognizes that the precise amount in a given dosage form may differ slightly from an intended amount for various reasons without materially affecting the in vivo effect of the administered compound.

The terms “comprising” and “including” are used herein in their open-ended and non-limiting sense unless otherwise noted.

By “a combination” or “in combination with” it is not intended to imply that the therapy or the therapeutic agents must be physically mixed or administered at the same time and/or formulated for delivery together, although these methods of delivery are within the scope described herein. A therapeutic agent in these combinations can be administered concurrently with, prior to, or subsequent to, one or more other additional therapies or therapeutic agents. The therapeutic agents can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized as single-agent therapeutics.

The combinations of the invention have therapeutic or protective functions or both. For example, these molecules may be administered to a human subject, to treat and/or prevent a variety of disorders, such as OA as described herein.

The term “combination” as used herein refers to either a fixed combination in one dosage unit form, or non-fixed combination, or a kit of parts for the combined administration where two or more therapeutic agents may be administered together, independently at the same time or separately within time intervals, especially where these time intervals allow that the combination partners show a cooperative, e.g., synergistic, effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., different i.a. formulations, or formulations for different routes of administration) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

By simultaneous therapeutic use, within the meaning of the present invention is meant an administration of at least two active ingredients by the same route and at the same time or at substantially the same time.

By separate use, within the meaning of the present invention is meant in particular an administration of at least two active ingredients at the same time or at substantially the same time by different routes.

By sequential therapeutic use is meant administration of at least two active ingredients at different times, the administration route being identical or different. More particularly by an administration method is meant according to which the whole administration of one of the active ingredients is carried out before administration of the other or others commences.

The terms “fixed combination”, “fixed dose” and “single formulation” as used herein refers to a single carrier or vehicle or dosage form formulated to deliver an amount, which is jointly therapeutically effective for the treatment of OA, of both therapeutic agents to a patient. The single vehicle is designed to deliver an amount of each of the agents along with any pharmaceutically acceptable carriers or excipients. In some embodiments, the vehicle is a solution or a suspension.

The term “non-fixed combination” or “kit of parts” means that the therapeutic agents of the combination of the invention are both administered to a patient as separate entities either simultaneously, concurrently or sequentially with no specific time limits, wherein such administration provides therapeutically effective levels of the two compounds in the body of a subject in need thereof.

### Compound 1 Demonstrates Therapeutic Efficacy

Compound 1 is a modified human ANGPTL3 protein that has been shown to demonstrate chondrogenic and chondroprotective effects. Compound 1 has been previously described in WO2014/138687, the contents of which are fully incorporated by reference. Without wishing to be bound by theory, it is believed that Compound 1 acts directly on cartilage resident-mesenchymal stromal cells (CR-MSCs) and articular chondrocytes through binding to α5β1 and αVβ3 integrins to transmit its anabolic repair effects on cartilage cells, promoting the formation of articular cartilage extracellular matrix proteins in mature chondrocytes and in CR-MSCs. The amino acid sequence of Compound 1 is set forth below in Table 1.

TABLE 1
Amino Acid Sequence of Compound 1

SEQ ID NO: 1
IPAECTTIYNRGEHTSGMYAIRPSNSQVFHVYCDVISGSPWTLIQHRID
GSQNFNETWENYKYGFGRLDGEFWLGLEKIYSIVKQSNYVLRIELEDWK
DNKHYIEYSFYLGNHETNYTLHLVAITGNVPNAIPENKDLVFSTWDHKA
KGHFNCPEGYSGGWWWHDECGENNLNGKYNKPRAQSKPERRRGLSWKSQ
NGRLYSIKSTKMLIHPTDSESFE

In some embodiments, the Compound 1 administered according to the described dosing regimens is unmodified. In other embodiments, the Compound 1 is PEGylated. In other embodiments, the Compound 1 is fused to a heterologous peptide. In certain embodiments, the Compound 1 is fused to any one of human serum albumin (HSA), an immunoglobulin heavy chain constant region (Fc), a polyhistidine, a glutathione S transferase (GST), a thioredoxin, a protein A, a protein G, a maltose binding protein (MBP), or a fragment of any of the foregoing heterologous polypeptide(s). In particular embodiments, the heterologous polypeptide is fused at the amino-terminal end of Compound 1. In additional or alternative embodiments, the heterologous polypeptide is fused at the carboxyl-terminal end of the Compound 1. In certain other embodiments, the Compound 1 is delivered according to the drug delivery system described in US Publication Number 2020/0108153, the contents of which are fully incorporated by reference herein.

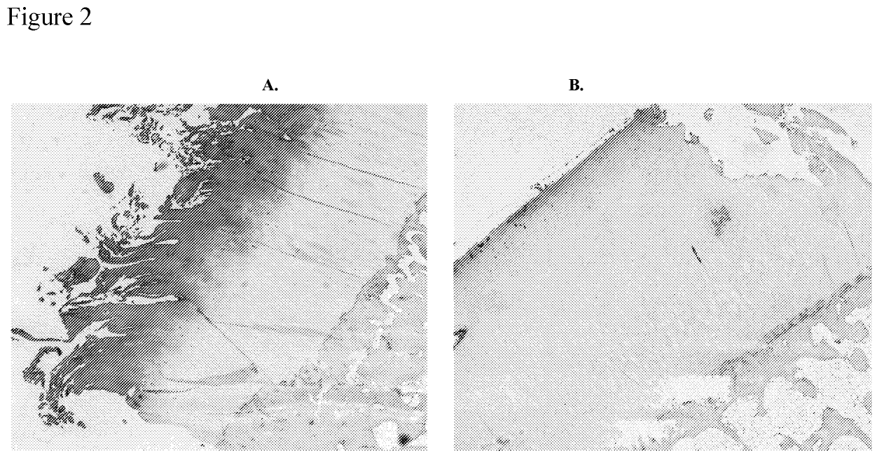

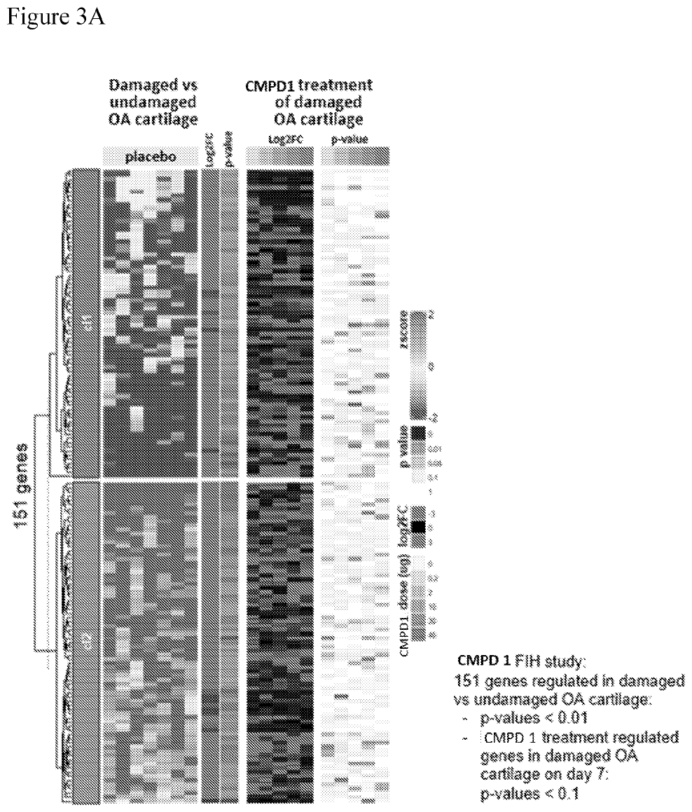

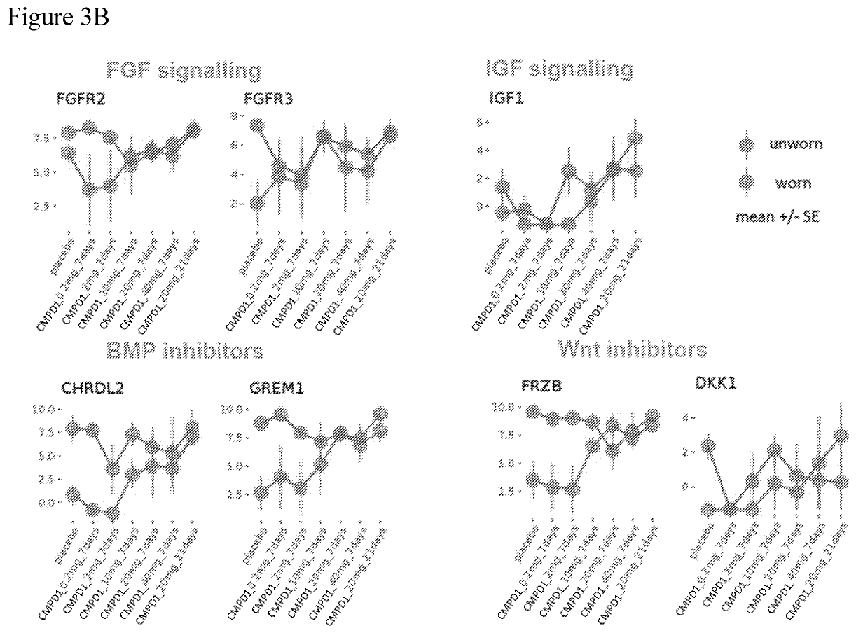

As explained above, Compound 1 has been shown to exhibit chondrogenic activity. For example, in a first-in-human (FIH) study Compound 1 was evaluated in human patients with OA who were scheduled for total knee replacement (TKR). Up to 40 mg Compound 1 was administered via intra-articular administration as a single dose 3 weeks, 1 week or 2 hours prior to surgery and safety, tolerability, pharmacokinetics (PK), and immunogenicity (IG) data were collected. Based on this study, it was determined that no significant drug-related Adverse Events (AE) or Serious Adverse Events (SAE) were reported, and Compound 1 was rapidly eliminated from the synovial fluid of the knee. Immunohistochemical analysis demonstrated that Compound 1 has a more pronounced penetration into damaged knee tissue as compared to undamaged tissue. (FIG. 2). RNA-Seq analysis also suggested Compound 1 modulates the activity of several genes involved in cartilage repair (FIG. 3A, B), and that this effect may last up to 21 days post-injection.

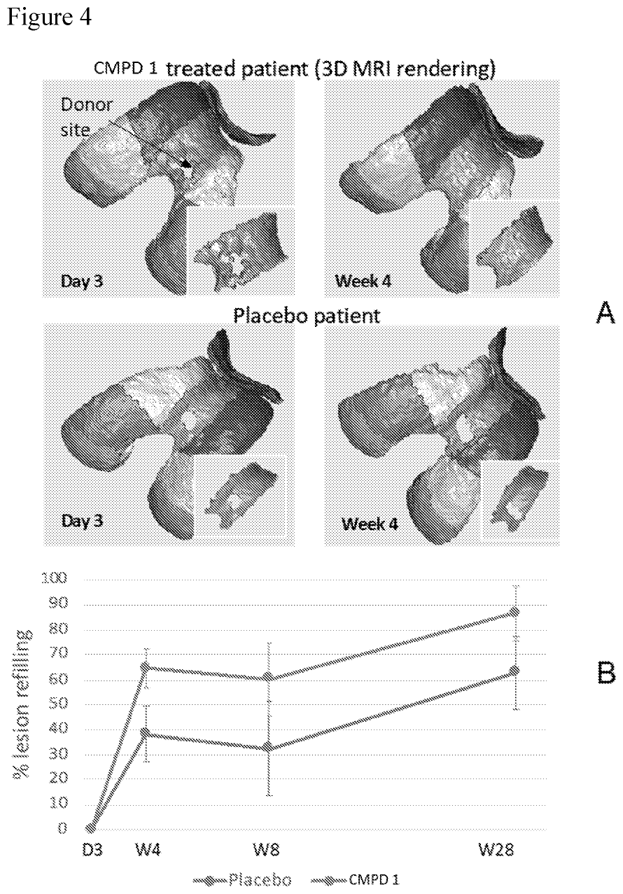

A proof of mechanism (PoM) study was subsequently performed in participants undergoing autologous chondrocyte implantation (ACI) to treat a focal cartilage lesion. Those participants received a single injection of 20 mg Compound 1 at the tissue-harvesting intervention, and both the extent of tissue growth and the quality of tissue composition were evaluated with 7 Tesla MRI including sodium sequences. Results showed that tissue compatible with early hyaline cartilage was detected at the donor sites at 4 and 12 weeks post-Compound 1 dose. (FIG. 4, 5). No drug-related safety signal, including hypersensitivity reaction, was reported. There were no deaths or SAEs during the study and all of the AEs reported were mild to moderate in severity.

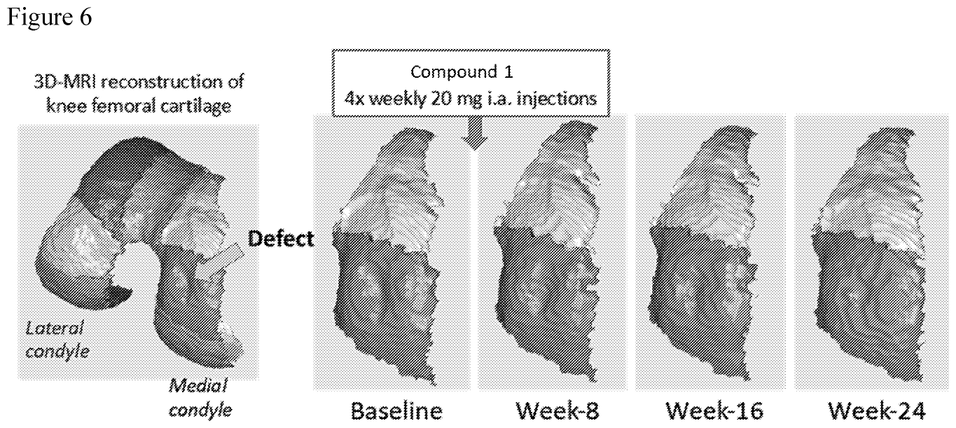

Part A of a proof of concept (PoC) study has completed dosing and follow-up in participants with knee cartilage injury who received 4 weekly injections of 20 mg Compound 1 and were then followed up for 52 weeks. Results confirm the cartilage anabolic activity of Compound 1 in humans at 28 weeks follow up measured with 3T MRI. (FIG. 6, 7). Overall, at the interim analysis, the treatment was well-tolerated and no relevant systemic safety signal was reported. Part B of the PoC study in participants with mild-moderate knee OA, receiving 4 monthly injections of 20 mg or 40 mg Compound 1, is currently ongoing.

The clinical data summarized above and described in more detail in the Examples affirm that Compound 1 demonstrates a clear dose response pattern coupled with a relatively short systemic exposure that surprisingly conveys a long pharmacodynamic effect. Compound 1 also demonstrates a favorable safety profile in human subjects. In view of this data, the inventors developed the dosing regimens disclosed herein as a means to convey a therapeutic benefit of Compound 1 while minimizing the number and frequency of injections. The disclosed dosing regimens are unexpectedly and surprisingly therapeutically effective even though the total number and frequency of doses is significantly less than previously thought necessary.

### Anti-IL-1β Antibodies

Intra-articular inflammation is driven by a plethora of pro-inflammatory cytokines (e.g., IL-1β, TNFα) and chemokines (e.g., CCL5, IL-8) that are present in joint tissues, especially synovium, synovial fluid and cartilage of OA patients. These inflammatory mediators downregulate cartilage matrix production by chondrocytes and increase production of matrix-degrading enzymes (MMPs, ADAMTS) by chondrocytes and synovial cells, leading to the breakdown and loss of the cartilage matrix. (van den Bosch 2019, Clin. Exp. Immunol. p. 153-166; Berenbaum 2013, Osteoarthr. Cartil. p. 16-21). IL-1β inhibits chondrocyte and progenitor cells anabolic activity and upregulates catabolic enzymes and osteogenic markers in vitro. In addition, strong evidence exists that IL-1 plays a critical role in OA (Wang et al 2015, Osteoarthr. Cartil. p. 22-30).

In accordance with the present invention, it has been surprisingly discovered that anti-IL-1β antibodies are useful in the treatment of patients with knee OA, and in particular knee OA with intra-articular inflammation. In one embodiment, the invention provides the use of an antibody which specifically binds to an IL-1β ligand or IL-1β receptor, preferably IL-1β ligand, in the prevention and/or treatment of knee OA with inflammation. Optionally, the antibody is a monoclonal antibody, humanized antibody, antibody fragment or single-chain antibody. In one aspect, the present invention concerns an isolated antibody which binds an IL-1β ligand. In another aspect, the antibody inhibits or neutralizes the activity of an IL-1β ligand (an antagonist antibody). In another aspect, the antibody is a monoclonal antibody, which has either human or non-human complementarily determining region (CDR) residues and human framework region (FR) residues. The antibody may be labeled and may be immobilized on a solid support. In a further aspect, the antibody is an antibody fragment, a monoclonal antibody, a single-chain antibody, or an anti-idiotypic antibody. In yet another embodiment, the present invention provides a composition comprising an anti-IL-1β ligand or IL-1β receptor antibody, preferably an anti-IL-1β ligand antibody, in a mixture with a pharmaceutically acceptable carrier. In one aspect, the composition comprises a therapeutically effective amount of the antibody. Preferably, the composition is sterile. The composition may be administered in the form of a liquid pharmaceutical formulation, which may be preserved to achieve extended storage stability. Alternatively, the antibody is a monoclonal antibody, an antibody fragment, a humanized antibody, or a single-chain antibody.

Anti-IL-1β antibodies that are suitable for use in the present invention and methods of preparing the same are described, for example, in U.S. Pat. Nos. 7,446,175 and 8,273,350, which are incorporated by reference in their entirety. An exemplary anti-IL-1β antibody suitable for use in the present invention is canakinumab. Other exemplary anti-IL-1β antibodies suitable for use in the present invention include gevokizumab and LY2189102. In another example, a soluble decoy receptor that is capable of binding IL-1β may be used. An example of a suitable decoy receptor is rilonacept.

### Methods of Treatment

Provided herein are methods of treating arthritis in a subject comprising administering to a joint of the subject an intra-articular dose of Compound 1 alone or in combination with an anti-IL-1β antibody. In some embodiments, the subject has arthritis, e.g., osteoarthritis, pre-arthritic trauma-associated changes, or autoimmune arthritis. In certain embodiments, the osteoarthritis is knee osteoarthritis. In further embodiments, the subject has osteoarthritis with inflammation. In other embodiments, the individual does not have, but is at risk for, arthritis with inflammation.

In embodiments where the disease or disorder is osteoarthritis, treatment according to one of the dosing regimens described herein is expected to slow or halt the progress of OA and reduce or eliminate symptoms associated with osteoarthritis as compared to treatment with placebo. In one non-limiting example, treatment may reduce pain as measured by the Knee Injury and Osteoarthritis Outcome Score (KOOS), the Western Ontario and McMaster Universities Osteoarthritis Index (WOMAC) score, or other art recognized methods of gauging pain reduction. In another embodiment, treatment may maintain or increase a patient's quality of life or activities of daily living as measured by the osteoarthritis knee and hip quality of life questionnaire (OAKHQOL), the WHO Quality of Life-BREF, Physical Activity Scale for the Elderly, or other art recognized methods. In another embodiment, treatment may result in the maintenance or regeneration of articular cartilage, ligaments, or tendons as measured using quantitative MRI, qualitative MRI, histology of biopsies, inspection during arthroscopy, or other art recognized methods of ascertaining changes in joint tissue. In other embodiments, treatment may result in the maintenance of joint structure based on cartilage volume as determined by quantitative MRI. In another non-limiting example, treatment according to one of the presently described dosing regimens may improve or maintain (e.g., prevent further decrease) the function in the affected joint as assessed through WOMAC function, KOOS score, reduction in stiffness, or other art recognized methods of assessing physical function. In other embodiments, increase in performance-based physical function can be assessed by a 40-meter (4×10 m) fast-paced walk test, 30-second chair stand test, 6-minute walking test, gait analysis, activity measures, or other art recognized methods. In another non-limiting example, treatment according to one of the presently described dosing regimens may prolong the survival of the joint affected with OA and/or increase the subject's quality of life. In yet another non-limiting example, treatment according to a dosage regimen of the present invention may prevent or delay the need for joint replacement surgery. In other embodiments, treatment may be effective in reducing synovitis or bursitis.

The therapeutic compounds may be administered according to any known administration method. In certain preferred embodiments, the therapeutic compounds are administered via intra-articular administration. Other possible routes of administration include, e.g., intradermal, intramuscular, intravenous, and sub-cutaneous. The therapeutic compounds may also be administered according to any known means for administering a therapeutic to a patient, including, but not limited to, a pre-filled syringe, a vial and syringe, an injection pen, an autoinjector, an i.v. drip and bag, a pump, a patch pump, etc. With such items, a patient may self-administer the drug (i.e., administer the drug on their own behalf) or a physician may administer the drug.

### Dosing Regimens

In some embodiments, Compound 1 and an anti-IL-1β antibody may be administered according to the dosage regimens described herein. The most effective dosages and dosage regimens for an individual subject may depend on the specific disease or condition to be treated and its severity. The dosing regimens may be continued or repeated until there is no longer a therapeutic benefit to the subject.

In some embodiments, the dosing regimen for Compound 1 may comprise one or more dosing cycles. Each dosing cycle may comprise one or more months, e.g., one month, two months, three months, four months, five months, six months, seven months, eight months, nine months, ten months, eleven months, or twelve months. A twelve month dosing cycle may also be referred to as a yearly dosing cycle. In preferred embodiments, the dosing cycle is a six-month or twelve-month (yearly) dosing cycle.

Each dosing cycle may comprise the administration of one or more, e.g., two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four, doses of Compound 1 during the dosing period. In embodiments comprising the administration of two or more doses during the dosing period, the doses may be administered during consecutive periods, e.g., consecutive days, weeks, or months. For example, in a dosing cycle comprising consecutive administration of four doses of Compound 1, each dose may be administered once a week for four consecutive weeks, or once a month for four consecutive months. Alternatively, the two or more doses may be administered in alternating periods of time, e.g., every other day, week, or month. For example, in a dosing cycle comprising alternate administration of four doses of Compound 1, each dose may be administered every two weeks (bi-weekly) for two months.

In some embodiments, each dosing cycle comprises the same number of doses. For example, in a dosing regimen comprising two dosing cycles, each dosing cycle may comprise three administrations of Compound 1. In other embodiments, the number of doses administered may vary from dosing cycle to dosing cycle within the same dosing regimen. For example, in a dosing regimen comprising three dosing cycles, dosing cycle 1 may comprise the administration of three doses of Compound 1 and dosing cycles 2 and 3 may comprise the administration of one dose of Compound 1.

In embodiments comprising two or more doses, the resting period does not begin until the final dose of the dosing period has been administered. For example, in a one month dosing cycle comprising two doses administered in alternate weeks, the doses could be administered in week one and week three, and the resting period could be week four. In some embodiments, the resting period is of the same period of time as the dosing period. For example, if the dosing period is one month then the resting period is one month. In other embodiments, the resting period is of a different period of time than the dosing period. For example, if the dosing period is one month then the resting period may be one week or two months. In some embodiments, the length of the dosing period and/or the length of the resting period may vary from dosing cycle to dosing cycle. For example, in a dosing regimen comprising two six-month dosing cycles, dosing cycle 1 may comprise a three-month dosing period and a three-month resting period, and dosing cycle 2 may comprise a one-month dosing period and a five-month resting period.

In a preferred embodiment, an Compound 1 dosing regimen comprises one or more six-month dosing cycles comprising one dose (i.e., a one-month dosing period) followed by a five-month resting period. In an alternative preferred embodiment, the six-month dosing cycle comprises three doses administered once a month for three consecutive months (i.e., a three-month dosing period) followed by a three-month resting period. In another preferred embodiment, the Compound 1 dosing regimen comprises one or more twelve-month dosing cycles comprising one dose (i.e., a one-month dosing period) followed by an eleven-month resting period. In an alternative preferred embodiment, a twelve-month dosing cycle comprises three doses administered once a month for three consecutive months (i.e., a three-month dosing period) followed by a nine-month resting period.

In one embodiment, an Compound 1 dosing regimen comprises at least four six-month dosing cycles, wherein each six-month dosing cycles comprises three doses of Compound 1 administered once a month for three consecutive months followed by a three-month rest period. In another embodiment, an Compound 1 dosing regimen comprises at least eight six-month dosing cycles, wherein dosing cycles 1-4 (i.e., the first four dosing cycles) comprise three doses of Compound 1 administered once a month for three consecutive months followed by a three-month rest period, and dosing cycles 5-8 comprise one dose of Compound 1 followed by a five-month rest period.

In another embodiment, an Compound 1 dosing regimen comprises four six-month dosing cycles, wherein each six-month dosing cycles comprises one dose of Compound 1 followed by a five-month rest period. In another embodiment, an Compound 1 dosing regimen comprises at least eight six-month dosing cycles, wherein each six-month dosing cycle comprises one dose of Compound 1 followed by a five-month rest period.

In another embodiment, an Compound 1 dosing regimen comprises at least two twelve-month dosing cycles, wherein three doses are administered once a month for three consecutive months followed by a nine-month resting period. In another embodiment, an Compound 1 dosing regimen comprises at least four twelve-month dosing cycles, wherein dosing cycles 1 and 2 comprise three doses are administered once a month for three consecutive months followed by a nine-month resting period, and dosing cycles 3 and 4 comprise one dose of Compound 1 followed by an eleven-month resting period.

In some embodiments, Compound 1 is administered in combination with an anti-IL-1β antibody. In some embodiments, the anti-IL-1β antibody is administered to the subject (e.g., a human subject) in a single injection. In some embodiments, the anti-IL-1β antibody is administered to the subject (e.g., human subject) in multiple injections. In some embodiments, the anti-IL-1β antibody is administered directly to the joint of a subject, e.g., intra-articular injection. In other embodiments, the anti-IL-1β antibody is administered to the patient systemically, e.g., subcutaneous, intravenous, or intra-muscular injection.

In some embodiments, the anti-IL-1β antibody is administered prior to the beginning of a Compound 1 dosage regimen. In some embodiments, the anti-IL-1β antibody is administered one week, two weeks, three weeks, four weeks, five weeks, six weeks, seven weeks, or eight weeks prior to administration of the first dose of Compound 1. In other embodiments the anti-IL-1β antibody is administered one month, two months, three months, four months, five months, or six months prior to administration of the first dose of Compound 1.

In other embodiments, the anti-IL-1 antibody is administered after beginning a Compound 1 dosage regimen. In some embodiments, the anti-IL-1β antibody is administered one week, two weeks, three weeks, four weeks, five weeks, six weeks, seven weeks, or eight weeks following administration of the first dose of Compound 1. In other embodiments the anti-IL-1β antibody is administered one month, two months, three months, four months, five months, or six months following administration of the first dose of Compound 1.

In another embodiment, the anti-IL-1β antibody is administered on the same day that the Compound 1 dosing regimen begins. In some embodiments, the anti-IL-1β antibody and Compound 1 are co-formulated and delivered in a single injection. In other embodiments, the anti-IL-1β antibody and Compound 1 are administered in separate injections.

The anti-IL-1β antibody may be administered one or more times during the course of the Compound 1 dosing regimen. In some embodiments, the anti-IL-1β antibody is administered once monthly, once every two months, once every three months, once every four months, once every five months, six months, or once yearly for a time period sufficient to treat the arthritis or cartilage damage with inflammation. In other embodiments, the anti-IL-1β antibody is administered one, two, three, four, five, or six times per dosing cycle. The effective dosages and the dosage regimens for Compound 1 and the anti-IL-1β antibody may be adjusted to provide the optimum desired therapeutic response depending on the subject and the disease or condition to be treated.

The timing of dosing in the dosing regimen is generally measured from the day of the first dose of the therapeutic compound. However, different health care providers use different naming conventions. Notably, week zero may be referred to as week 1 by some health care providers, while day zero may be referred to as day one by some health care providers. Thus, it is possible that different physicians will designate, e.g., a dose as being given during week 3/on day 21, during week 3/on day 22, during week 4/on day 21, during week 4/on day 22, while referring to the same dosing schedule. For consistency, the first week of dosing will be referred to herein as week 1, while the first day of dosing will be referred to as day 1. However, it will be understood by a skilled artisan that this naming convention is simply used for consistency and should not be construed as limiting, i.e., weekly dosing is the provision of a weekly dose of the therapeutic compound regardless of whether the physician refers to a particular week as “week 1” or “week 2”. It will further be understood that a dose need not be provided at an exact time point, e.g., a dose due approximately on day 29 could be provided, e.g., on day 24 to day 34, e.g., day 30, as long as it is provided in the appropriate week. Moreover, a “monthly dose” can be provided four to five weeks after the preceding dose.

### Dosage Amounts

In some embodiments, the dose of Compound 1 administered via intra-articular injection is about 10-100 mg, about 10-90 mg, about 10-80 mg, about 10-70 mg, about 10-60 mg, about 10-50 mg, about 10-40 mg, about 10-30 mg, about 10-20 mg, about 20-100 mg, about 20-90 mg, about 20-80 mg, about 20-70 mg, about 20-60 mg, about 20-50 mg, about 20-40 mg, about 20-30 mg, about 30-100 mg, about 30-90 mg, about 30-80 mg, about 30-70 mg, about 30-60 mg, about 30-50 mg, about 30-40 mg, about 40-100 mg, about 40-90 mg, about 40-80 mg, about 40-70 mg, about 40-60 mg, about 40-50 mg, about 50-100 mg, about 50-90 mg, about 50-80 mg, about 50-70 mg, about 50-60 mg, about 60-100 mg, about 60-90 mg, about 60-80 mg, about 60-70 mg, about 70-100 mg, about 70-90 mg, about 70-80 mg, about 80-100 mg, about 80-90 mg, or about 90-100 mg.

In other embodiments, the dose of Compound 1 administered via intra-articular injection is about 10-55 mg, about 10-45 mg, about 10-35 mg, about 10-25 mg, about 10-15 mg, about 15-60 mg, about 15-55 mg, about 15-50 mg, about 15-45 mg, about 15-40 mg, about 15-35 mg, about 15-30 mg, about 15-25 mg, about 15-20 mg, about 20-55 mg, about 20-45 mg, about 20-35 mg, about 20-25 mg, about 25-60 mg, about 25-55 mg, about 25-50 mg, about 25-45 mg, about 25-40 mg, about 25-35 mg, about 25-30 mg, about 30-55 mg, about 30-45 mg, about 30-35 mg, about 35-60 mg, about 35-55 mg, about 35-50 mg, about 35-45 mg, about 35-40 mg, about 40-55 mg, about 40-45 mg, about 45-60 mg, about 45-55 mg, about 45-50 mg, about 50-55 mg, or about 55-60 mg.

In other embodiments, the dose of Compound 1 administered via intra-articular injection is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In preferred embodiments, the dose of Compound 1 administered is about 20-40 mg, about 20 mg, or about 40 mg.

In some embodiments, the dose of Compound 1 administered via intra-articular injection is 10-100 mg, 10-90 mg, 10-80 mg, 10-70 mg, 10-60 mg, 10-50 mg, 10-40 mg, 10-30 mg, 10-20 mg, 20-100 mg, 20-90 mg, 20-80 mg, 20-70 mg, 20-60 mg, 20-50 mg, 20-40 mg, 20-30 mg, 30-100 mg, 30-90 mg, 30-80 mg, 30-70 mg, 30-60 mg, 30-50 mg, 30-40 mg, 40-100 mg, 40-90 mg, 40-80 mg, 40-70 mg, 40-60 mg, 40-50 mg, 50-100 mg, 50-90 mg, 50-80 mg, 50-70 mg, 50-60 mg, a 60-100 mg, 60-90 mg, 60-80 mg, 60-70 mg, 70-100 mg, 70-90 mg, 70-80 mg, 80-100 mg, 80-90 mg, or 90-100 mg.

In other embodiments, the dose of Compound 1 administered via intra-articular injection is 10-55 mg, 10-45 mg, 10-35 mg, 10-25 mg, 10-15 mg, 15-60 mg, 15-55 mg, 15-50 mg, 15-45 mg, 15-40 mg, 15-35 mg, 15-30 mg, 15-25 mg, 15-20 mg, 20-55 mg, 20-45 mg, 20-35 mg, 20-25 mg, 25-60 mg, 25-55 mg, 25-50 mg, 25-45 mg, 25-40 mg, 25-35 mg, 25-30 mg, 30-55 mg, 30-45 mg, 30-35 mg, 35-60 mg, 35-55 mg, 35-50 mg, 35-45 mg, 35-40 mg, 40-55 mg, 40-45 mg, 45-60 mg, 45-55 mg, 45-50 mg, 50-55 mg, or 55-60 mg.

In other embodiments, the dose of Compound 1 administered via intra-articular injection is 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. In preferred embodiments, the dose of Compound 1 administered is 20-40 mg, 20 mg, or 40 mg.

The anti-IL-1β antibody can be administered via intra-articular injection at a dose of about 150-1000 mg. In some embodiments, the intra-articular dose of an anti-IL-1β antibody is about 150-800 mg. In some embodiments, the intra-articular dose of an anti-IL-1β antibody is about 150-600 mg. In some embodiments, the intra-articular dose of an anti-IL-1β antibody is about 300-600 mg. In some embodiments, the intra-articular dose of an anti-IL-1β antibody is about 450-600 mg. In some embodiments, the intra-articular dose of an anti-IL-1β antibody is about 450-800 mg. In some embodiments, the intra-articular dose of an anti-IL-1β antibody is about 150 mg. In some embodiments, the intra-articular dose of an anti-IL-1β antibody is about 300 mg. In some embodiments, the intra-articular dose of an anti-IL-1β antibody is about 450 mg. In some embodiments, the intra-articular dose of an anti-IL-1β antibody is about 600 mg. In some embodiments, the intra-articular dose of an anti-IL-1β antibody is about 750 mg. In some embodiments, the intra-articular dose of an anti-IL-1β antibody is about 800 mg. In some embodiments, the intra-articular dose of an anti-IL-1β antibody is about 1000 mg.

The anti-IL-1β antibody can be administered via intra-articular injection at a dose of 150-1000 mg. In some embodiments, the intra-articular dose of an anti-IL-1β antibody is 150-800 mg. In some embodiments, the intra-articular dose of an anti-IL-1β antibody is 150-600 mg. In some embodiments, the intra-articular dose of an anti-IL-1β antibody is 300-600 mg. In some embodiments, the intra-articular dose of an anti-IL-1β antibody is 450-600 mg. In some embodiments, the intra-articular dose of an anti-IL-1β antibody is 450-800 mg. In some embodiments, the intra-articular dose of an anti-IL-1β antibody is 150 mg. In some embodiments, the intra-articular dose of an anti-IL-1β antibody is 300 mg. In some embodiments, the intra-articular dose of an anti-IL-1β antibody is 450 mg. In some embodiments, the intra-articular dose of an anti-IL-1β antibody is 600 mg. In some embodiments, the intra-articular dose of an anti-IL-1β antibody is 750 mg. In some embodiments, the intra-articular dose of an anti-IL-1β antibody is 800 mg. In some embodiments, the intra-articular dose of an anti-IL-1β antibody is 1000 mg.

### Kits

The disclosure also encompasses kits for treating a patient with OA. In some embodiments, the kits are for treating a patient with knee OA. In other embodiments, the kits are for treating a patient with knee OA with inflammation. Such kits comprise Compound 1 (e.g., in liquid or lyophilized form) or a pharmaceutical composition comprising Compound 1 and one o more pharmaceutically acceptable carriers. In some embodiments, such kits further comprise an anti-IL-1β antibody, such as canakinumab. Additionally, such kits may comprise means for administering the Compound 1 or the anti-IL-1β antibody (e.g., a syringe and vial, a prefilled syringe, a prefilled pen, a patch/pump) and instructions for use. The instructions may disclose providing the Compound 1 and/or the anti-IL-1β antibody to the patient as part of a specific dosing regimen.

The phrase “means for administering” is used to indicate any available implement for systemically administering a drug to a patient, including, but not limited to, a pre-filled syringe, a vial and syringe, an injection pen, an autoinjector, an i.v. drip and bag, a pump, patch/pump, etc. With such items, a patient may self-administer the drug (i.e., administer the drug on their own behalf) or a care-giver or a physician may administer the drug.

Disclosed herein are kits for the treatment of a patient having knee osteoarthritis with or without intra-articular inflammation, comprising: (a) a pharmaceutical composition comprising a therapeutically effective amount of Compound 1 and one or more pharmaceutically acceptable carriers; b) means for administering the Compound 1; and (c) instructions for the intra-articular administration of Compound 1.

Disclosed herein are kits for the treatment of a patient having knee osteoarthritis with or without intra-articular inflammation, comprising: (a) a pharmaceutical composition comprising a therapeutically effective amount of Compound 1 and/or a therapeutically effective amount of an anti-IL-1β antibody, and one or more pharmaceutically acceptable carriers; (b) means for administering the Compound 1 and/or anti-IL-1β antibody; and (c) instructions for the intra-articular administration of Compound 1 and/or the anti-IL-1β antibody.

In one specific embodiment, a use is provided, of (a) a pharmaceutical composition comprising Compound 1 and one or more pharmaceutically acceptable carriers, (b) a pharmaceutical composition comprising an anti-IL-1β antibody and one or more pharmaceutically acceptable carriers, and (c) means for intra-articular administration of the Compound 1 and anti-IL-1β antibody to a patient having knee OA with or without intra-articular inflammation wherein:
i) the anti-IL-1β antibody is to be administered intra-articularly to the patient with a dose of about 150 to about 600 mg, on day 1 of treatment; and
ii) the Compound 1 is to be administered intra-articularly to the patient with a dose of about 20 to 40 mg two weeks after the injection of the anti-IL-1 antibody; and,
iii) thereafter, the Compound 1 is to be administered intra-articularly to the patient with a dose of about 20 to 40 mg at four week (monthly) intervals according to a dosage regimen disclosed herein.

In another specific embodiment, a use is provided, of a) a pharmaceutical composition comprising Compound 1, b) a pharmaceutical composition comprising an anti-IL-1β antibody, and c) means for intra-articular administration of the Compound 1 and anti-IL-1β antibody to a patient having knee OA with or without intra-articular inflammation wherein:
i) the anti-IL-1β antibody is to be administered intra-articularly to the patient with a dose of 600 mg, on day 1 of treatment; and
ii) the Compound 1 is to be administered intra-articularly to the patient with a dose of 40 mg two weeks after the injection of the anti-IL-1β antibody; and,
iii) thereafter, the Compound 1 is to be administered intra-articularly to the patient with a dose of 40 mg at four week (monthly) intervals for a total of three doses.

In a further embodiment, the invention concerns an article of manufacture, comprising: (a) a composition comprising an anti-IL-1β antibody; (b) a container containing said composition; and (c) a label affixed to said container, or a package insert included in said container referring to the use of said anti-IL-1β antibody in the treatment of knee OA with inflammation. The composition may comprise a therapeutically effective amount of an anti-IL-1β antibody.

In yet a further embodiment, the invention provides a method or use as defined above, comprising co-administration of a therapeutically effective amount of Compound 1, preferably in a pharmaceutically acceptable delivery form such as intraarticularly, intravenously or subcutaneously, and a second drug substance, said second drug substance being an anti-inflammatory compound in free form or salt form.

### EXAMPLES

The following examples are intended to illustrate the invention and are not to be construed as being limitations thereon. Abbreviations are used as is conventional in the art.

### Example 1: A Randomized, Placebo Controlled, Double-Blind First-In-Human Single Ascending Dose Study of Compound 1 in Primary Osteoarthritis Patients Scheduled for Total Knee Replacement

Methods: A first-in-human, randomized, single-center, double-blind, placebo-controlled, single ascending dose trial was conducted in patients aged 50-75 years with knee OA scheduled for total knee replacement (TKR). Patients were randomized 3:1 (Compound 1 to placebo) in each of 7 cohorts consisting of 4 patients each. The 5 increasing intra-articular dose levels ranging 0.2-40 mg (0.2 mg, 2 mg, 10 mg, 20 mg, and 40 mg) were administered 7 days before TKR. Two additional 20 mg dose levels were also administered 2 hours or 21 days before TKR. Key safety parameters included AEs, injection-site reactions and detection of anti-drug antibodies against Compound 1. Knee tissues were obtained during the TKR procedure to assess local exposure to Compound 1 through immunohistochemical (IHC) staining, and RNA sequence (RNA-Seq) analysis was performed on cartilage tissue originating from visually damaged or undamaged areas of the joint surface that was removed intraoperatively.

Results: In total, 30 patients were randomized to Compound 1 (n=21) or placebo (n=7). Two patients withdrew consent prior to treatment. The mean age of recruited patients was 63 years, 68% were female (n=19), and 96% were caucasian (n=27). A total of 19 (Compound 1, n=14; placebo, n=5) patients experienced at least one AE. The overall incidence of AEs was 66.7% (14/21) for Compound 1 and 71.4% (5/7) for placebo. One drug-related case of dry mouth/dysgeusia was reported in the 40 mg cohort, which resolved spontaneously and was considered mild. Ten SAEs were reported in five patients (Compound 1, n=3; placebo, n=2), although these were considered related to the surgery and not related to Compound 1. Anti-Compound 1 antibodies were not detected in any patient. After intra-articular injection, Compound 1 was dose-dependently distributed from the joint to systemic circulation with Cₘₐₓ typically reached between 2 to 6 hours after the administration, followed by rapid elimination. IHC demonstrated that Compound 1 penetrated the articular cartilage shortly after injection (2 hours), with more pronounced penetration into the damaged areas (FIG. 2). Compound 1 was not detectable in the articular cartilage or synovial-fluid 7 days post intra-articular injection (after administration of up to 20 mg).

RNA-Seq analysis demonstrated that 151 genes were significantly up- or down-regulated in damaged versus undamaged articular cartilage samples from placebo-treated OA patients (FIG. 3A). Compound 1 counter-regulated most of these OA-regulated genes in damaged cartilage 7 days post treatment, suggesting a broad effect of Compound 1 on genes involved in OA pathogenesis. RNA-Seq analysis further demonstrated modulation of several genes involved in cartilage homeostasis and repair, suggesting a broad effect by Compound 1 up to 21 days post-injection (FIG. 3B). These effects were both dose-dependent and mostly present in the damaged cartilage tissue.

Conclusions: In this study, Compound 1 displayed a favorable safety profile without any clinically significant drug-related safety signals or immunogenicity. Compound 1 further showed a tendency to preferentially penetrate into damaged cartilage; was quickly cleared locally and systemically; and counter-regulated several cartilage genes involved in OA pathogenesis at the RNA level.

### Example 2: A Randomized, Placebo-Controlled, Patient and Investigator Blinded, Single Dose, Proof of Concept Study Investigating the Safety, Tolerability and Preliminary Efficacy of Intra-Articular Compound 1 in Regenerating the Articular Cartilage of the Knee at Donor Sites in Patients Undergoing Autologous Chondrocyte Implantation

Methods: This was a randomized, placebo-controlled, double-blind, single dose, proof-of-mechanism study in subjects with cartilage lesions undergoing autologous chondrocyte implantation (ACI). In total, 14 subjects were treated with a single i.a. injection (9 in Compound 1 20 mg and 5 in placebo, 2:1 randomization ratio) that was administered at the end of the first surgical procedure. The study was designed to assess cartilage regeneration 1) at the artificially created ACI donor site in the intercondylar notch with a full thickness cartilage defect and 2) at the index lesion cartilage lesions (defect site). Spontaneous repair was minimized by avoiding breaching the bone lamina while performing the biopsy. Assessments of the treatment effects were performed at Day 3 (baseline), Week 4 (primary endpoint), Week 12 and Week 28 using 7T MRI to detect early signs of cartilage matrix production both at the donor site and the defect site, along with histological confirmation at week 4 (2^{nd} ACI treatment arthroscopy). The index lesion to be treated with ACI was assessed with MRI only at Day 3 and Week 4, prior to the implantation of the chondrocyte graft. Volumes of the donor site and the cartilage sub-region containing the main lesion, as well as their glycosaminoglycan content (GAG), were measured by 7 Tesla high-resolution morphological (proton)-MRI and indirectly by sodium-MRI, respectively. While the volume of the donor site was measured via manual segmentation of the 3D proton images, the cartilage sub-region volume containing the main lesion, whose shape is by nature more complex than the surgically created lesion, was measured via an automated segmentation approach using a 3D-active shape model. All sodium-MRI measurements were performed using a 15-channel sodium-only knee array coil and images with a resolution of 1.5×1.5×3 mm³ were obtained in 25 min of scanning time. For region of interest (ROI) analyses, sodium concentration maps were rescaled to the resolution of morphological proton images and overlaid with the corresponding morphological image. Cartilage sodium concentrations were calculated by using a calibration curve obtained for each scan from agarose phantoms having different sodium concentrations. GAG content in the index region was normalized to that of corresponding healthy regions of the same knee. During the second surgical procedure on Week 4, a biopsy of the regenerated tissue was taken at the donor site, and tissue debris from the defect site was collected for histological and immunohistochemical analysis prior to the ACI graft implantation.

Results: The i.a. injection of Compound 1 resulted in a 65±8% refilling (vs placebo: 38±11%, p=0.04) of the donor site after 4 weeks in all treated patients (FIG. 4A) and increased to 86±11% at Week 28 (vs placebo: 63±14%, p=0.12) (FIG. 4B). In two placebo patients, a partial refilling of the donor site was seen at Week 4, but it was not maintained at Week 12 and therefore considered a blood clot after a potential lesion in the osseous lamina that had been absorbed at Week 12. Similarly, partial repair of the main cartilage lesion was observed at Week 4, prior to the ACI graft implantation (change from baseline in the volume of the sub-region encompassing the defect—Compound 1: +128±97 mm³ vs placebo: +16±30 mm³, p=0.03).

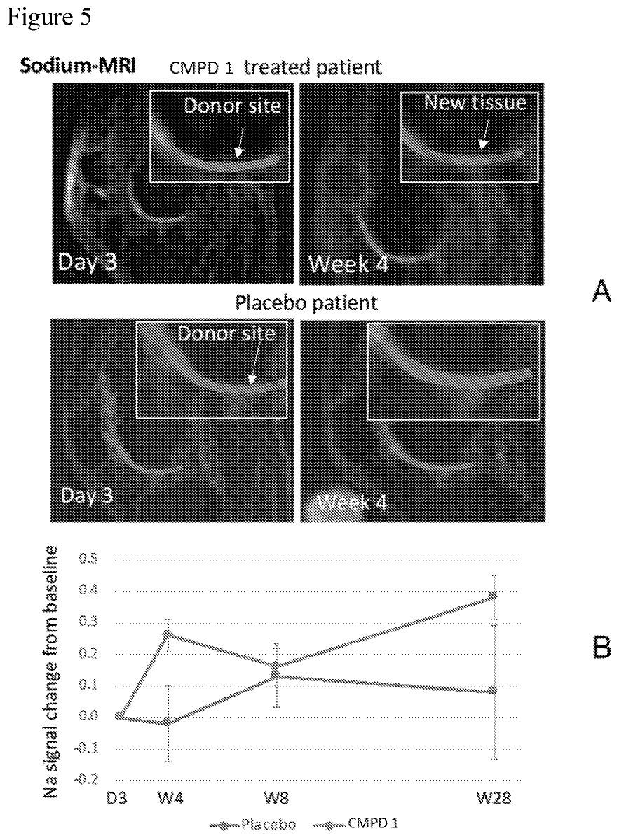

Sodium-MRI confirmed the hyaline-like cartilage nature of the regenerated tissue in the donor site: The sodium signal in the donor site increased by 26±5%, 16±6% and 38±7% in the Compound 1 group vs. −2±12% (p=0.12), 13±10% (p=0.51) and 8±21% (p=0.15) in the placebo group at Weeks 4, 12 and 28, respectively indicating increasing GAG content in the Compound 1 group (FIG. 5B). Post-hoc pooled analysis of the sodium MRI data from both the donor and defect sites showed a statistically significant (p=0.01) increase in sodium signal intensity at Week 4.

Histological and immunohistochemical assessments of biopsies taken at the donor site on Week 4 early demonstrated features of hyaline cartilage in the regenerated tissue of Compound 1—treated patients, as suggested by semi-quantitative International Cartilage Regeneration & Joint Preservation Society (ICRS) II histological scoring, and by collagen type 2 staining.

Compound 1 was rapidly distributed from the joint to the systemic circulation and no drug-related AEs or SAEs were reported during the course of this study.

Conclusions: A single i.a. injection of Compound 1 at 20 mg promoted refilling of the biopsy donor site with a full thickness cartilage defect in patients undergoing an ACI procedure. The newly regenerated cartilage tissue at the donor site appeared of hyaline-like quality as evidenced from its enriched content in proteoglycans detected by sodium MRI (FIG. 5A). Exploratory assessment of the index lesion, prior to grafting 4 weeks after the i.a. injection of Compound 1, also showed signs of tissue formation from filling of the lesion. Finally, Compound 1 displayed a consistent systemic pharmacokinetic profile together with a favorable safety profile with no significant drug related safety signals and no immunogenicity.

### Example 3: A Two-Part, Randomized, Placebo-Controlled, Patient and Investigator Blinded, Study Investigating the Safety, Tolerability and Preliminary Efficacy of Intra-Articular Compound 1 Injections in Regenerating the Articular Cartilage of the Knee in Patients with Articular Cartilage Lesions (Part A) and in Patients with Knee Osteoarthritis (Part B)

Part A—Methods: This is a randomised, double-blind, placebo (“PBO”)-controlled, proof-of-concept study in patients with a partial thickness cartilage lesion. 58 patients (43 [20 mg Compound 1]; 15 [PBO]), stratified by lesion type (condylar or patellar) were treated with 4 weekly i.a. injections. The primary endpoint was T2 relaxation time measurement as a marker of collagen fiber network, cartilage lesion-volume was a secondary endpoint, both using 3 Tesla MRI. Assessments were performed at baseline, weeks (wks) 8, 16, 28 and 52 (the last in 23/58 patients). While lesion volume was determined from manually segmented images, the cartilage volume of 21 sub-regions spanning the entire knee was measured from 3D isotropic MR images employing an automated segmentation software (MR Chondral Health [MRCH], Siemens). The treatment effect was evaluated for the index region volume encompassing the lesion (FIG. 6).

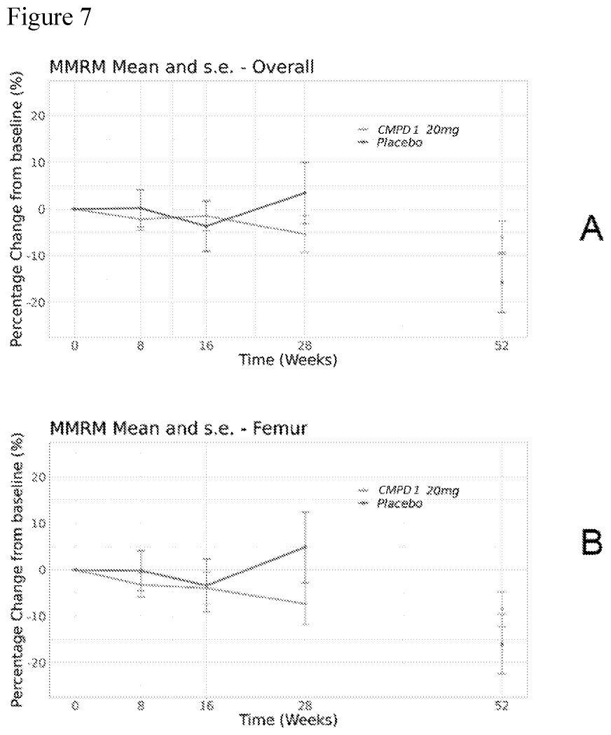

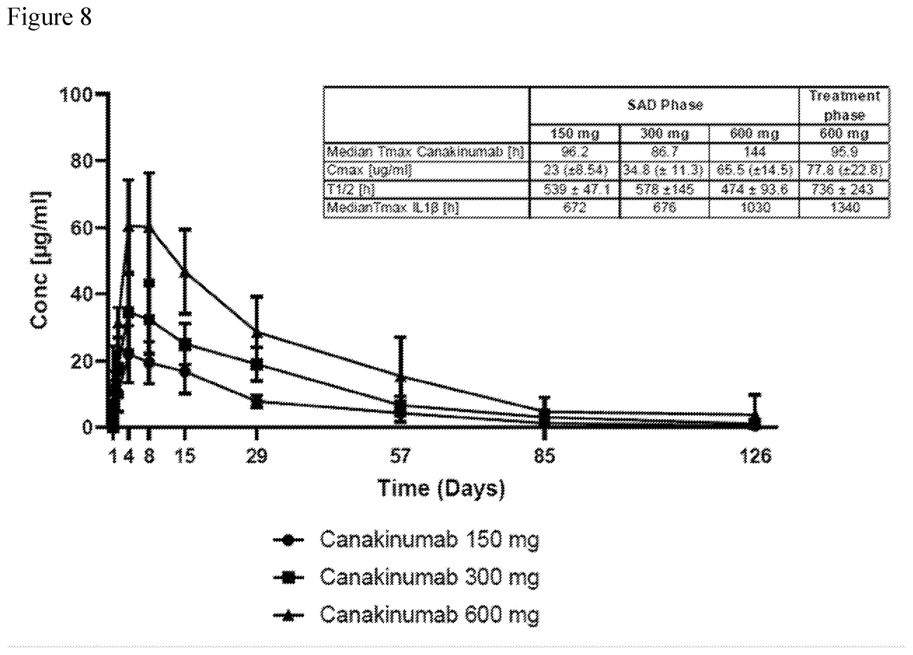

Part A—Results: There was a reduction in the cartilage defect volume at EoS (week 28), measured with high-resolution MRI (manual segmentation), in response to Compound 1. Such reduction was even more pronounced if the percentage change from baseline in cartilage defect volume was used as the response variable in the MMRM model (FIG. 7). Particularly, the one-sided p-value related to the difference between Compound 1 and placebo at week 28 was 0.08 (vs. PBO) for the sub-group of patients with a femoral lesion (the p-values at weeks 16 and 53 were 0.47 and 0.85 respectively). In contrast, no sign of defect filling could be detected in the subgroup of patients with a patellar lesion, no change in T2 relaxation time values was detected between treatment and PBO groups. Given the limitations of measuring small, irregularly-shaped lesions with manual image-analysis, the MRCH approach was used (FIG. 6) and Compound 1-induced refilling of the cartilage lesions in patients with femoral lesions was detected (Δ=96 mm³ at wk 16). Limiting the analysis to patients with condylar lesions only, the benefit of Compound 1 appeared to be maintained at wks 28 (Δ=68 mm³) and 52 (Δ=117 mm³). No overgrowth was detected in the lateral femoral condyles without cartilage damage.

The overall safety profile was positive with treatment emergent mild/moderate local reactions (incidence of joint swelling [9.3% vs 0%] and arthralgia [7.0% vs 6.7%] for Compound 1 vs PBO) resolving spontaneously or with paracetamol/NSAIDs. No anti-drug antibodies were detected.

Part A—Conclusion: Treatment with 4 weekly i.a. injections of 20 mg Compound 1 resulted in regeneration of damaged cartilage in patients with femoral articular cartilage lesions. Automated measurement of cartilage volume in the femoral index region was able to detect a relevant treatment effect and was found to be more sensitive than the manual segmentation method. No sign of cartilage overgrowth was observed in healthy femoral regions. Compound 1 showed a favorable safety and tolerability profile.

Part B—Methods: This is a randomised, double-blind, placebo (PBO)-controlled, proof-of-concept study in patients with mild to moderate osteoarthritis (Kellgren and Lawrence (K&L) grade 2-3 and joint space width 2-4 mm). 75 patients (25 [40 mg Compound 1], 25 [20 mg Compound 1]; 25 [PBO]), are treated with a total of 4 i.a. injections over the period of 4 months. The primary endpoint is safety and tolerability as well as the change in cartilage volume/thickness in the index region at Week 28 and 52 using 3 Tesla MRI. Cartilage quality will be assessed as secondary endpoint using T2 relaxation times as surrogate marker. In addition, pain and function are evaluated using the KOOS as secondary endpoints. Further exploratory endpoints comprise PK/PD assessments, biomarker, protein-expression and gene analyses. Assessments are performed at baseline, weeks 8, 16, 28 and 52. While lesion volume is determined from manually segmented images, the cartilage volume of 21 sub-regions spanning the entire knee is measured from 3D isotropic MR images employing an automated segmentation software (MR Chondral Health [MRCH], Siemens). The treatment effect is evaluated for the index region volume encompassing the lesion.

Part B—Results: Cartilage thickness and volume are expected to increase compared to baseline in the treatment groups, while a stable course or deterioration is expected in placebo patients. Pain and function are expected to improve. Data is currently insufficient to predict a dose dependent effect for Compound 1. The safety profile so far is favorable.

### Example 4: Intra-Articular Canakinumab, an Anti-IL-1β Antibody in for the Treatment of Painful Knee Osteoarthritis: A Randomized, Double Blind, Placebo and Naproxen Controlled Phase I/II Study

To assess the effect of intra-articular injection of an anti-IL-1β antibody for the treatment of knee OA with inflammation, the following clinical study was designed and conducted. This was a phase II, randomized, double-blind, placebo and naproxen controlled, multicenter study.

The study consisted of two phases:
SAD (single ascending dose) phase, assessing the safety and tolerability of up to four doses of canakinumab ranging from 150 mg through 300 mg to 600 mg.
Treatment phase, assessing the effects of a single i.a. dose of canakinumab on self-reported pain with reference to placebo (primary objective) and an active control, naproxen 2×500 mg daily (exploratory objective) utilizing a double-blind, double-dummy, parallel group design.

Methods: The study included patients with mild to moderate osteoarthritis (K&L 2-3). For eligibility, patients had to discontinue all NSAID or other analgesic medication 24 hours before randomization (SAD) or at least five half-lives (3-7 days) before their randomization (Treatment). Patients also had to report moderate to severe pain (intensity between 40 to 100 mm on the VAS (visual analogue scale) in the index knee during the last 24 hours, and confirm that they experienced pain on most days during the past month.

Results: Twenty-four patients were enrolled in the SAD study and 145 patients in the Treatment study. All patients completed the SAD study (100%), while 120 of the 145 enrolled patients (82%) completed the Treatment study. Discontinuation rates were similar across treatment arms (canakinumab 9, placebo 7, naproxen 9 patients). Release of canakinumab into the systemic circulation was rapid with detectable concentration (i.e. >0.2 μg/mL) apparent as early as 1 hour following the injection. Canakinumab as i.a. injection was generally safe and well tolerated. Overall incidence of AEs was higher in the canakinumab (35, 77.8%) and the naproxen (37, 69.8%) groups compared with the placebo (27, 57.4%) group. Infections and infestations were the most common AEs reported in all groups. The frequency of infections and infestations AEs was somewhat higher in the canakinumab (33.3%) and the naproxen (32.1%) groups compared with the placebo (21.3%) group. Gastrointestinal AEs were less frequently reported in the canakinumab (15.6%) group compared with the naproxen group (25.4%), while frequency in the placebo was 21.3%. There were no deaths reported during either study phase. Only one SAE was associated with canakinumab in the SAD Phase (celluitis) and none in the Treatment phase: All others were reported by patients receiving either placebo (2 SAEs by 1 patient in the SAD Phase, 4 SAEs by 4 patients in the Treatment Phase) or Naproxen (6 SAEs reported by 4 patients in the Treatment Phase). None of the SAEs was suspected as related to study medication by either the investigator or the Novartis safety expert.

Circulating levels of hs-CRP were measured in all available exploratory samples collected during the Treatment phase to examine the anti-inflammatory effect of canakinumab. Data were available for 113/145 patients with measurable levels and no relevant protocol deviations. Focusing on patients with elevated hs-CRP levels, 72.2% (13/18) responded with a drop below the 2 mg/L threshold in the canakinumab group; the corresponding numbers for the placebo and naproxen groups were only 16.6% (3/18) and 33.3% (8/24), respectively. The decreases in hs-CRP from baseline (%) were calculated from log-transformed values. The responses to canakinumab were markedly greater than responses to either placebo or naproxen (>0.001). There was no significant correlation between hs-CRP and VAS pain levels or WOMAC Pain at baseline.

Conclusions: The study demonstrated a significant anti-inflammatory effect of treatment with canakinumab in patients with OA. The clinical benefits of IL-1β inhibition was variable. The safety profile of canakinumab compared to an active comparator was good.

### Example 5: A Randomized, Four-Arm, Placebo-Controlled, Participant, Investigator and Sponsor-Blinded Study Investigating the Safety, Tolerability and Efficacy of Intra-Articular Canakinumab Followed by Intra-Articular Compound 1 in Patients with Knee Osteoarthritis

The purpose of this study is to assess the efficacy of intra-articular injection of Compound 1 alone, or in combination with an anti-IL-1β antibody, in patients with symptomatic, knee OA with inflammation. This study is designed to demonstrate that dosing of Compound 1 alone, or in combination with an anti-IL-1β antibody, results in the regeneration of articular cartilage tissue in patients with symptomatic knee OA with inflammation. This study also aims to demonstrate the ability of Compound 1 alone, or in combination with an anti-IL-1β antibody, to reduce pain and/or inflammation in patients with symptomatic knee OA with inflammation.

Methods: This is a non-confirmatory, randomized, four arm, placebo-controlled, participant, investigator and sponsor blinded (with regards to canakinumab whereas Compound 1 treatment is open label) study in patients with knee OA with inflammation. Participants are eligible with mild to moderate (K&L 2-3), suffering moderate to severe OA pain (corresponding to NRS (numeric rating scale) Pain≥5 to ≤9) in the target knee for the majority of days in the last 3 months prior to screening, an hsCRP≥2 mg/L, and contrast-enhanced MRI (CE-MRI) diagnosed moderate or severe knee synovitis based on an established synovititis scoring system (moderate score 9-12 or severe score≥13) (Guermazi et al 2011).

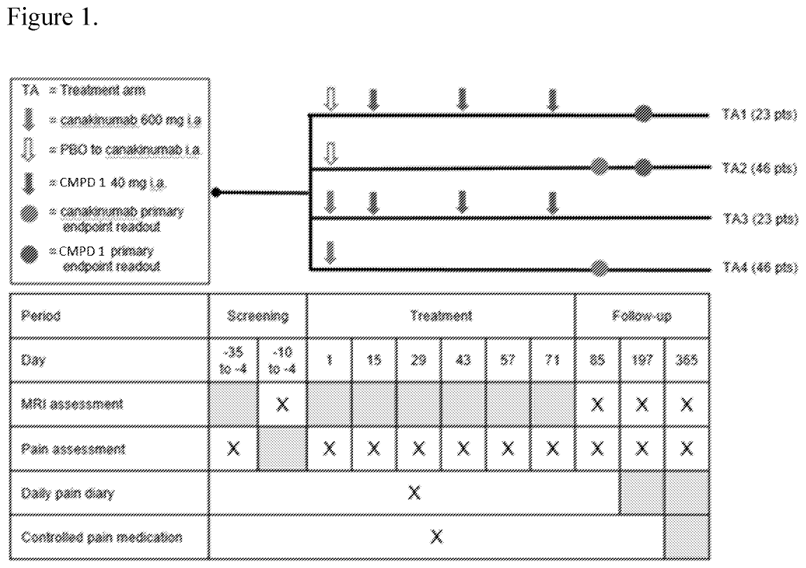

Participants will be randomly assigned to one of the following 4 Treatment Arms (TA) in a ratio of 1:2:1:2 (FIG. 1):
TA1: Single i.a. injection of placebo to canakinumab followed by q4w×3 i.a. injections of 40 mg Compound 1
TA2: Single i.a. injection of placebo to canakinumab
TA3: Single i.a. injection of 600 mg canakinumab followed by q4w×3 i.a. injections of 40 mg Compound 1
TA4: Single i.a. injection of 600 mg canakinumab

Participants will receive an i.a. injection of either canakinumab 600 mg, or matching placebo. Fourteen days later, participants randomized to treatment arms 1 and 3 will receive i.a. injections of Compound 1 40 mg every 4 weeks, on Day 15, 43 and 71, with two intermediate follow up remote visits, on Day 29 and 57. Participants randomized to TA 2 and 4 will attend the same visits but will not receive further study drug. The canakinumab treatment is placebo controlled and blinded to participants and investigators whereas the Compound 1 treatment is open label.

Clinical experience to date with Compound 1 indicates that this compound demonstrates an acceptable safety profile and is well-tolerated. A dose of 40 mg has been selected for repeat dosing, for three doses, every four weeks (q4w×3), based on safety and feasibility, and in order to maximize the likelihood of delivering a sustained, pharmacodynamic effect leading to cartilage repair in the knee. The dose of canakinumab given in this study will be 600 mg, as a single i.a. injection as that dosage has previously been shown to be well tolerated (See Example 4).

The primary endpoints in the study are the change in KOOS Pain subscale at Day 85 as efficacy parameter for canakinumab. For the efficacy of Compound 1 the change in cartilage volume in the index region measured by MRI at Day 197 will be assessed. The secondary endpoints related to safety, PK, immunogenicity, pain, structure, inflammation and function at other time points.

Results: The study has two primary objectives. First, assess the efficacy of q4w×3 i.a. injections of Compound 1 vs. no injections of Compound 1, in maintaining or regenerating articular cartilage tissue. This objective will be determined by measuring the change from baseline in cartilage volume in the index region by MRI at Day 197. It is anticipated that treatment with Compound 1 will result in the maintenance or regeneration of articular cartilage tissue. Second, assess the efficacy of a single i.a. injection of canakinumab vs. placebo in relieving OA pain. This objective will be determined by measuring the change from baseline KOOS Pain subscale at Day 85. It is anticipated that treatment with canakinumab will relieve OA pain.

The study will also assess the efficacy of each treatment arm in meeting several secondary objectives:
The efficacy of a single i.a. injection of canakinumab followed by q4w×3 i.a. injections of Compound 1 vs. a single i.a. injection of canakinumab vs. only q4w×3 ia. injections of Compound 1 in regenerating articular cartilage and will be assessed. This objective will be determined by measuring the change in cartilage volume and thickness of the index region by MRI at Day 197 and 365. It is anticipated that treatment with a single i.a. injection of canakinumab, a single i.a. injection of canakinumab followed by q4w×3 i.a. injections of Compound 1, and q4w×3 i.a. injections of Compound 1 will be effective in regenerating articular cartilage.
The efficacy of a single i.a. injection of canakinumab vs. placebo to canakinumab, on synovitis will be assessed. This objective will be determined by measuring the change in synovitis level measured from Ktrans by DCE-MRI at Day 85. It is anticipated that a single i.a. injection of canakinumab as compared to placebo will be effective in reducing synovitis.
The efficacy of a single i.a. injection of canakinumab vs. placebo to canakinumab in relieving OA pain and improving function over time will be assessed. This objective will be determined by measuring the change in numeric rating scale (NRS) Pain at Day 15, 29, 43, 57, 71 and 85, and the change in KOOS Pain and Function in daily living (ADL) subscales at Day 15, 29, 43, 57, 71 and 85. It is anticipated that a single i.a. injection of canakinumab as compared to placebo will be effective at relieving OA pain and improving function over time.
The chondro-anabolic effects of Compound 1 and the potential for synergistic effects with canakinumab will be assessed. This objective will be determined by assessing cartilage formation and degeneration biomarkers in synovial fluid, such as but not limited to Type 2 collagens (PIIBNP, PIIANP) and Hyaluronic Acid at Day 1, 15, 43 and 71. It is anticipated that Compound 1 will display a chondro-anabolic effect and will interact synergistically with canakinumab.

Having thus described several aspects of several embodiments, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

Those skilled in the art will recognize, or be able to ascertain, using no more than routine experimentation, numerous equivalents to the specific embodiments described specifically herein. Such equivalents are intended to be encompassed in the scope of the following claims.

1. A method of treating knee osteoarthritis with intra-articular inflammation, the method comprising administering to a human subject in need thereof one or more doses of a therapeutically effective amount of Compound 1 by intra-articular injection to the knee joint of the subject according to a dosing regimen comprising one or more dosing cycles.

2. The method of claim 1, wherein said dosing cycle is a six-month dosing cycle comprising three intra-articular injections per dosing cycle with one injection administered once a month for three consecutive months.

3. The method of any of claims 1-2, wherein the amount of Compound 1 administered per dose is 40 mg.

4. The method of any of claims 1-3, wherein said dosing regimen comprises at least two dosing cycles.

5. The method of any of claims 1-4, wherein treatment according to said dosing regimen results in the maintenance or regeneration of articular cartilage tissue as determined by MRI analysis.

6. A method of treating knee osteoarthritis with intra-articular inflammation, the method comprising administering to a human subject in need thereof one or more doses of a therapeutically effective amount of an anti-inflammatory antibody by intra-articular injection to a knee joint of the subject.

7. The method of claim 6, wherein the anti-inflammatory antibody is an anti-IL-1β antibody.

8. The method of any of claims 6-7, wherein the anti-inflammatory antibody is canakinumab.

9. The method of any of claims 6-8, wherein the amount of the anti-inflammatory antibody administered per dose is 600 mg.

10. The method of any of claims 6-9, wherein administration of the anti-inflammatory antibody results in a reduction in OA pain.

11. A method of treating knee osteoarthritis with intra-articular inflammation, the method comprising administering to a joint of a human subject in need thereof:
(a) a dose of a therapeutically effective amount of an anti-IL-1β antibody, and
(b) one or more doses of a therapeutically effective amount of Compound 1 according to a dosing regimen comprising one or more dosing cycles,
wherein said dose of the anti-IL-1β antibody and said one or more doses of a therapeutically effective amount of Compound 1 are administered by intra-articular administration.

12. The method of claim 11, wherein the dose of the anti-IL1β antibody is administered prior to beginning the Compound 1 dosing regimen.

13. The method of any of claims 11-12, wherein the anti-IL1β antibody is administered two weeks prior to beginning the Compound 1 dosing regimen.

14. The method of any of claims 11-13, wherein the anti-IL-1β antibody is administered four weeks prior to the one or more doses of Compound 1.

15. The method of claims 11-14, wherein the therapeutically effective amount of an anti-IL-1β antibody is 600 mg.

16. The method of any of claims 11-15, wherein said dosing cycle is a six-month dosing cycle comprising three intra-articular injections administered once a month for three consecutive months.

17. The method of any of claims 11-16, wherein the amount of Compound 1 administered per dose is 40 mg.

18. The method of any of claims 11-17, wherein said dosing regimen comprises at least two dosing cycles.

19. The method of any of claims 11-18, wherein a dose of 600 mg of an anti-IL-1β is administered prior to beginning the second dosing cycle of Compound 1.

20. The method of any of claims 11-19, wherein said administration results in the maintenance or regeneration of articular cartilage tissue as determined by MRI analysis.

21. The method of any of claims 11-19, wherein said administration results in a reduction in OA pain.
